(19) United States
(12) Patent Application Publication
(10) Pub. No.: US 2009/0105674 A1
(43) Pub. Date: Apr. 23, 2009

(54) SNIVEL ASPIRATOR

(76) Inventor: I-Jung Cheng, Tainan City (TW)

Correspondence Address: I-Jung Cheng, P. O. BOX 90, Tainan City, 70499, TW

(21) Appl. No.: 11/874,940

(22) Filed: Oct. 19, 2007

(51) Int. Cl. A61M 1/00

(52) U.S. Cl. 604/320

(57) ABSTRACT

A snivel aspirator includes a bottle and a snivel aspirator main body. The bottle has a bottle lid having an air-aspirating tube communicating with the bottle and having a non-return valve. The bottle lid further has an air-sucking tube communicating with the bottle and disposed with an air-valve switch. The snivel aspirator main body connected with the air-sucking tube of the bottle has a filth-collecting box and an air-outgoing passageway communicating with the air-sucking tube of the bottle by a connecting tube, disposed with an air-valve press button fixed with an insert rod having a spring fitted thereon and having its lower end secured with a clogging block facing the air-outgoing passageway. The snivel aspirator main body has another side provided with a snivel-aspirating tube communicating with the filth-collecting box.

### BACKGROUND OF THE INVENTION

1. Field of the Invention

This invention relates to a snivel aspirator, particularly to one composed of a bottle and a snivel aspirator body, labor-saving, convenient and safe in use and able to let a nurse avoid inhaling a patient's snivel and getting co-infection.

2. Description of the Prior Art

As commonly known, mucous membrane in a man's respiratory tract has mucous glands and cilia, and the mucus secreted by the mucous glands will be actuated by swinging of the cilia to move backward to a man's throat to be swallowed unconsciously; therefore, a man's nasal cavity can produce a cleaning and moistening effect. When a man's respiratory tract is infected with germs, the mucus secreted by the mucous gland will reduce and become thick, and the cilia will be destroyed by virus or germs and unable to swing normally. Thus, harmful substance in the respiratory tract cannot be removed out and germs are likely to grow and multiply. Since babies or patients need to be taken care of by nurses and unable to blow the nose by themselves; therefore, snivel will be clogged in the nasal cavity to cause hard breathing. A conventional snivel aspirator (A1), as shown in FIG. 1, includes a bottle (A11) and bottle lid (A12) threadably assembled on the bottle (A11) and provided thereon a snivel-sucking tube (A13) and an air-sucking tube (A14). In using, firstly, the snivel-sucking tube (A13) has one end inserted in a patient's nostril and then a nurse inhales air through the air-sucking tube (A14) for sucking out snivel from the patient's nostril. However, during aspirating snivel with the conventional snivel aspirator (A1), it is hard to control suction force evenly and smoothly. In case of sucking with a comparatively small force, snivel will be unable to be sucked out of the nostril, but sucking with too great a force may let a nurse inhale the patient's snivel and cause co-infection.

### SUMMARY OF THE INVENTION

The objective of this invention is to offer a snivel aspirator labor-saving, convenient and safe in use and able to let a nurse avoid inhaling a patient's snivel and getting co-infection.

The snivel aspirator in the present invention includes a bottle and a snivel aspirator main body. The bottle is provided with a bottle lid and has one side disposed with an air-aspirating tube communicating with the bottle and having a non-return valve installed in the interior. The bottle has another side provided with an air-sucking tube communicating with the bottle and having an air-valve switch assembled thereon. The snivel aspirator main body connected with the air-sucking tube of the bottle is assembled with a filth-collecting box and has one side disposed with an air-outgoing passageway communicating with the air-sucking tube of the bottle by a connecting tube. The snivel aspirator main body is further provided thereon with an air-valve press button fixed with an insert rod inserted through the snivel aspirator main body and fitted thereon with a spring. The insert rod has its underside secured with a clogging block corresponding to the air-outgoing passageway. The snivel aspirator main body further has another side disposed with a snivel-aspirating tube communicating with the filth-collecting box.

### BRIEF DESCRIPTION OF DRAWINGS

This invention will be better understood by referring to the accompanying drawings, wherein:

FIG. 1 is an exploded perspective view of a conventional snivel aspirator;

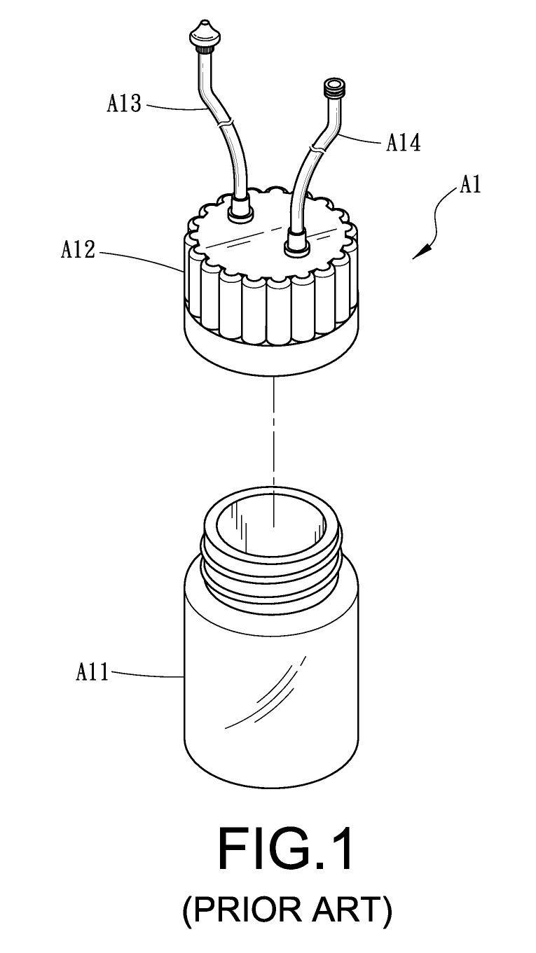

FIG. 2 is a cross-sectional view of a snivel aspirator in the present invention;

FIG. 3 is a cross-sectional view of the snivel aspirator in a first operating condition in the present invention; and

FIG. 4 is a cross-sectional view of the snivel aspirator in a second operating condition in the present invention.

### DETAILED DESCRIPTION OF THE PREFERRED EMBODIMENT

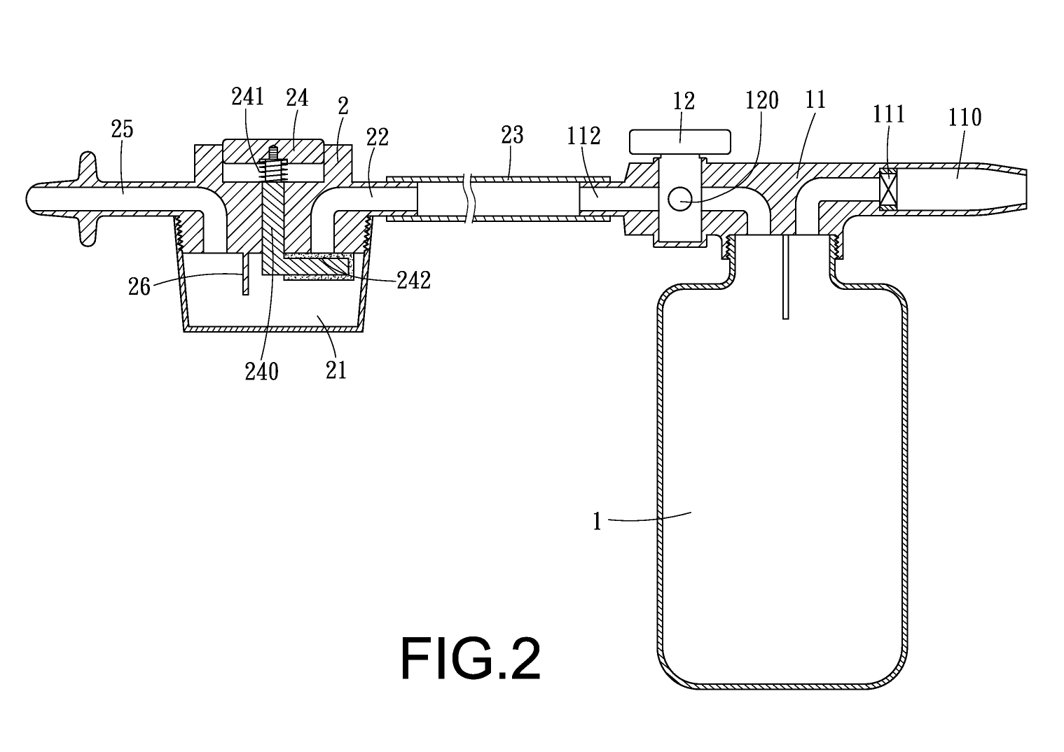

A preferred embodiment of a snivel aspirator in the present invention, as shown in FIG. 2, includes a bottle 1 and a snivel aspirator main body 2 combined together.

The bottle 1 is provided with a bottle lid 11 having one side connected with an air-aspirating tube 110, communicating with the bottle 1 and having a one-way non-return valve 111 disposed therein. The bottle lid 11 has the other side provided with an air-sucking tube 112 communicating with the bottle 1 and having an air-valve switch 12 assembled thereon for opening and closing the passageway of the air-sucking tube 112. The air-valve switch 12 is bored with a transverse through hole 120.

The snivel aspirator main body 2 is connected with the air-sucking tube 112 of the bottle 1 and threadably assembled with a filth-collecting box 21. The snivel aspirator main body 2 has one side provided with an air-outgoing passageway 22 communicating with the filth-collecting box 21 and connected with a connecting tube 23 to communicate with the air-sucking tube 112 of the bottle 1. Further, the snivel aspirator main body 2 is assembled thereon with an air-valve press button 24 having an insert rod 240 inserted through the snivel aspirator main body 2. The insert rod 240 is fitted thereon with a spring 241 and has its lower end fixed with a clogging block 242 for blocking the air-outgoing passageway 22. Furthermore, the snivel aspirator main body 2 has the other side disposed with a snivel-aspirating tube 25 communicating with the filth-collecting box 21, having its underside fixed with a separating plate 26 extending in the filth-collecting box 21 for blocking snivel and letting it deposited in the filth-collecting box 21.

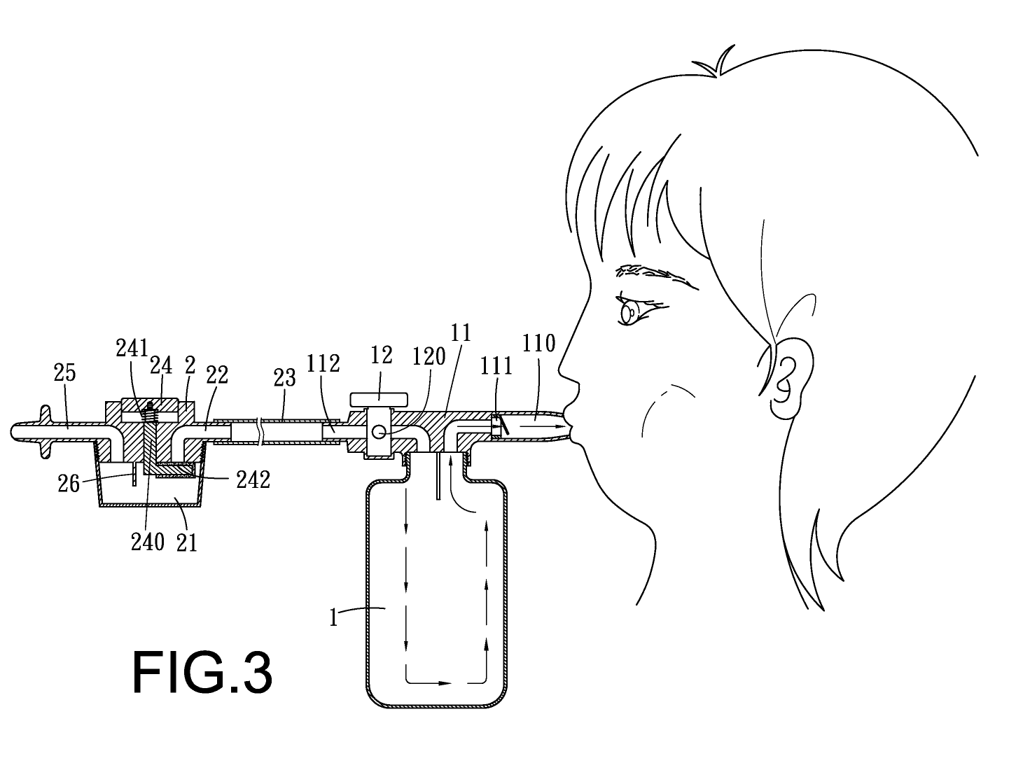

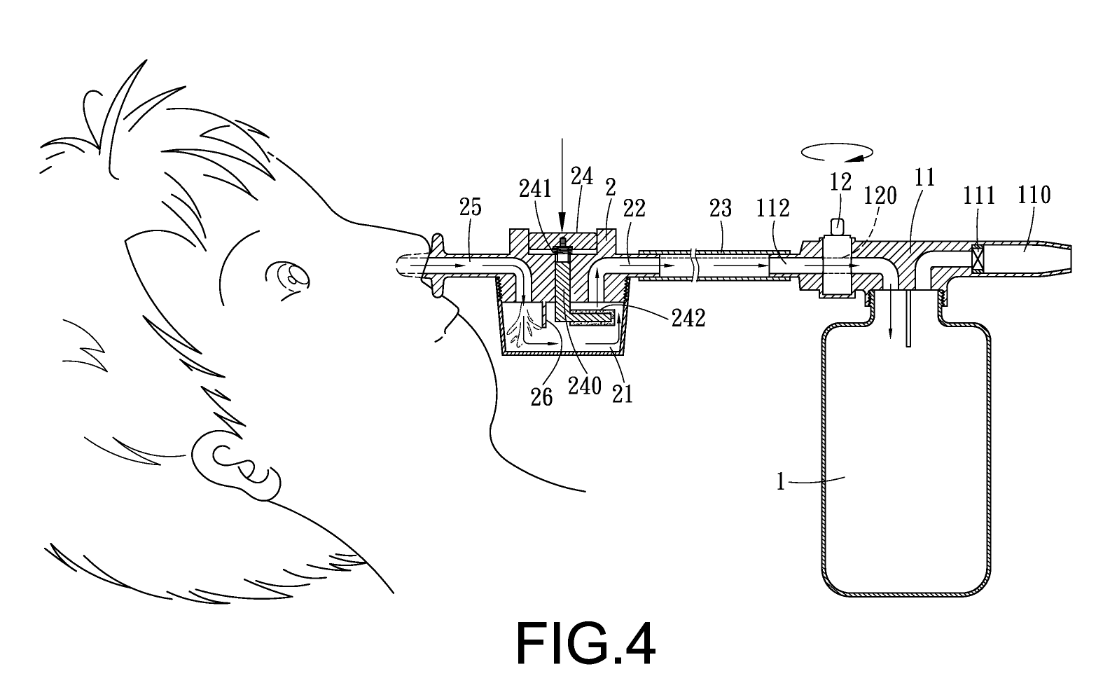

In using and operating, referring to FIGS. 3 and 4, the air-valve switch 12 on the bottle 1 is first closed and air in the bottle 1 is sucked out through the air-aspirating tube 110 to let the interior of the bottle 1 be in a vacuum condition. To carry out aspirating snivel, firstly, insert the snivel-aspirating tube 25 of the snivel aspirator main body 2 in a patient's nostril and then open the air-valve switch 12 on the bottle 1 to let the through hole 120 of the air-valve switch 12 communicate with the air-sucking tube 112. Next, press the air-valve press button 24 on the snivel aspirator main body 2 to drive the insert rod 240 to move downward and actuate the clogging block 242 at its lower end to move away from the air-outgoing passageway 22, and thus snivel can be aspirated out of the patient's nostril by the vacuum action of the bottle 1 and the snivel aspirated out will be blocked by the separating plate 26 and flow into the filth-collecting box 21.

As can be understood from the above description, this invention has the following advantages.

1. During aspirating and removing snivel, a nurse can avoid inhaling a patient's snivel and getting co-infection.

2. The separating plate 26 of the snivel aspirator main body 2 can block off snivel to let it deposited in the filth-collecting box 21, which can conveniently be disassembled for washing clean to insure safety in use.

3. A patient's snivel is aspirated and removed out by suction force of the vacuum bottle so snivel aspirating work can be carried out evenly, smoothly and conveniently.

4. The condition of snivel aspirating and removing can be checked conveniently.

While the preferred embodiment of the invention has been described above, it will be recognized and understood that various modifications may be made therein and the appended claims are intended to cover all such modifications that may fall within the spirit and scope of the invention.

What is claimed is:

1. A snivel aspirator comprising:
a bottle having a bottle lid assembled thereon, said bottle lid having one side disposed with an air-aspirating tube communicating with said bottle, said air-aspirating tube provided with a non-return valve in the interior, said bottle lid having another side disposed with an air-sucking tube communicating with said bottle, said air-sucking tube assembled thereon with an air-valve switch; and
a snivel aspirator main body connected with said air-sucking tube of said bottle, said snivel aspirator main body provided with a filth-collecting box, said snivel aspirator main body having one side provided with an air-outgoing passageway, said air-outgoing passageway connected with a connecting tube communicating with said air-sucking tube of said bottle, said snivel aspirator main body mounted thereon with an air-valve press button, said air-valve press button fixed with an insert rod inserted through said snivel aspirator main body, said insert rod fitted thereon with a spring, said insert rod having its lower end secured with a clogging block corresponding to said air-outgoing passageway, said snivel aspirator main body having another side provided with a snivel-aspirating tube communicating with said filth-collecting box.

2. The snivel aspirator as claimed in claim 1, wherein said air-valve switch of said bottle is bored with a through hole.

3. The snivel aspirator as claimed in claim 1, wherein said snivel aspirator main body has its underside fixed with a separating plate extending in said filth-collecting box.
